(19) United States
(12) Patent Application Publication
(10) Pub. No.: US 2024/0238544 A1
(43) Pub. Date: Jul. 18, 2024

(54) POSITIVE END EXPIRATORY PRESSURE RETENTION VALVE

(71) Applicant: Michael S. Jarrett, Huntingdon, TN (US)

(72) Inventor: Michael S. Jarrett, Huntingdon, TN (US)

(21) Appl. No.: 18/575,831

(22) PCT Filed: Jul. 5, 2022
(86) PCT No.: PCT/US22/36156

§ 371 Date: Dec. 30, 2023

Related U.S. Application Data
(60) Provisional application No. 63/218,172, filed on Jul. 2, 2021.

(51) Int. Cl. A61M 16/00; A61M 16/04; A61M 16/08; A61M 16/20; A61M 39/22

(52) U.S. Cl. CPC A61M 16/0087; A61M 16/0402; A61M 16/0816; A61M 16/201; A61M 16/208; A61M 39/227; A61M 2205/3331

(57) ABSTRACT

A positive end expiratory pressure retention valve has a tube house forming an air flow chamber between a first port connected to a medical air supply tube and a second port connected to a medical air supply source. A balloon house is connected to the tube house and houses a balloon in a balloon chamber connected to the air flow chamber. The balloon occludes the air flow chamber when inflated.

### CROSS-REFERENCE TO RELATED APPLICATIONS

This application is a U.S. National Stage of International Application No. PCT/US22/36156 filed Jul. 5, 2022 which claims the benefit of U.S. Provisional Patent Application Ser. No. 63/218,172 filed Jul. 2, 2021, all of which are hereby incorporated by reference in their entirety.

### FIELD

This disclosure relates generally to the design, manufacture, and use of medical fluid lines. More specifically, this disclosure is related to positive end expiratory pressure retention valve systems.

### BACKGROUND

Currently, retention (i.e., maintaining) positive end expiratory pressure (PEEP) for medical patients is particularly problematic, for example in ventilator based care for patients receiving care with high positive end expiratory pressure (PEEP) requirements using an endotracheal tube (ETT), when the ETT is removed from the attached ventilator and placed on another device there is nothing to maintain PEEP and prevent aerosolization of potentially infectious airborne particles. Traditionally, in this instance retaining PEEP is accomplished by the extra step of clamping the ETT with hemostats prior to pressure source (e.g., ventilator) removal. However this is not without risk as this method has caused holes in the ETT, which results in a need to exchange the tube, resulting in loss of PEEP and aerosolization of potentially infectious airborne particles. Additionally, the clamping method both weakens the tube and has caused the ETT adapter to come out of the ETT, again resulting in a loss of PEEP and aerosolization of airborne particles.

### SUMMARY

According to one aspect of the disclosed subject matter, a positive end expiratory pressure retention valve is provided. The positive end expiratory pressure retention valve has a tube house forming an air flow chamber between a first port connected to a medical air supply tube and a second port connected to a medical air supply source. A balloon house is connected to the tube house and houses a balloon in a balloon chamber connected to the air flow chamber. The balloon occludes the air flow chamber when inflated.

These and other aspects of the disclosed subject matter, as well as additional novel features, will be apparent from the description provided herein. The intent of this summary is not to be a comprehensive description of the claimed subject matter, but rather to provide a short overview of some of the subject matter's functionality. Other systems, methods, features and advantages here provided will become apparent to one with skill in the art upon examination of the following FIGUREs and detailed description. It is intended that all such additional systems, methods, features and advantages that are included within this description, be within the scope of any claims.

### BRIEF DESCRIPTION OF THE FIGURES

The features, natures, and advantages of the disclosed subject matter may become more apparent from the detailed description set forth below when taken in conjunction with the drawings in which like reference numerals indicate like features.

FIGS. 1, 2, and 3 are diagrams showing an embodiment of a PEEP retention valve having a deflated balloon.

FIGS. 4, 5, and 6 are diagrams showing the embodiment of the PEEP retention valve of FIGS. 1, 2, and 3 and having an inflated balloon.

FIG. 7 is a diagram of an embodiment of a balloon housing.

FIGS. 8 and 9 are diagrams of the balloon housing of FIG. 7 showing the air intake of a balloon rolled/stretched over the ribs and the fillable body of the in the balloon housing.

FIGS. 10 and 11 are diagrams showing a cap for a balloon port.

FIG. 12 is a diagram showing a cap on and over the balloon housing of FIG. 8.

FIG. 13 is a diagram showing a cap on the PEEP retention valve of FIG. 1.

FIG. 14 is a diagram showing a PEEP retention valve consistent and attached to a ventilator circuit WYE.

FIGS. 15, 16, and 17 are diagrams of a PEEP retention valve adapter consistent.

FIG. 18 is a diagram showing a side perspective view of the PEEP retention valve of FIG. 1 connected to an ETT.

FIGS. 19 through 22 are diagrams showing the flow of inflation of a in a positive end expiratory pressure retention valve.

### DESCRIPTION

The following description is not to be taken in a limiting sense, but is made for the purpose of describing the general principles of the present disclosure. The scope of the present disclosure should be determined with reference to the claims. Exemplary embodiments of the present disclosure may be illustrated in the drawings, like numbers being used to refer to like and corresponding parts of the various drawings. The dimensions of drawings provided are not shown to scale.

The innovations described provide a solution for the loss of positive end expiratory pressure (PEEP) and aerosolization of airborne particles in medical tubes primarily such as endotracheal tubes (ETT).

The positive end expiratory pressure valve solutions provided are described with reference to a three port adapter valve that attaches directly to a medical air supply tube, for example an in-use endotracheal tube (ETT). The valve works (e.g., retains positive end expiratory pressure) by inflating a balloon, for example made of neoprene or non-latex rubber like material (latex being critically dangerous for patients with latex allergies) which occludes, in an airtight seal, the lumen of the air flow chamber from the ETT. By inflating the balloon and occluding (i.e., blocking) the lumen of the air flow chamber from the ETT, the patient retains positive end expiratory pressure PEEP when disconnected from the ventilator circuit while allowing for connection/reconnection if needed. Airborne pathogens are also prevented from exiting the ETT and potentially infecting caregivers during ventilator disconnection.

Advantageously, the valve may be a small lightweight value. The valve may also be used as a disposable respiratory supply item. The valve may be manufactured through the process of injection molding or 3D printing and made of a material such as plastic, silicone, or neoprene. The valve is designed to fit onto the end of a medical tube, such as an endotracheal tube upon endotracheal intubation, and coupled to a high pressure source, such as a ventilator. The valve may fit onto existing ETT adapters for ventilator to ETT connection or may be manufactured as an ETT adapter for ventilator to ETT.

The valve has a 15 mm to 22 mm diameter adaptor housing a balloon in the center. The balloon is designed to be inflated any time the patient needs to be removed from the ventilator or bag valve device and placed on another device. Inflation and deflation of the balloon may be accomplished with a 10 cc syringe. Inflation of the balloon anytime the patient is removed from the vent or other device will retain positive end expiratory pressure (PEEP) and prevent aerosolization of potentially airborne pathogens.

Thus, the valve solution provided retains PEEP in patients that require high levels of PEEP, for example due to Acute Respiratory Distress Syndrome (ARDS). Patients may develop ARDS for a variety of reasons: Pneumonia, Flu, Aspiration, Coronavirus and other reasons. Many of the reasons patients develop ARDS are due to contagious infectious processes. The valve solution and corresponding balloon inflation PEEP retention will prevent these airborne particles from being passed on to nearby caregivers.

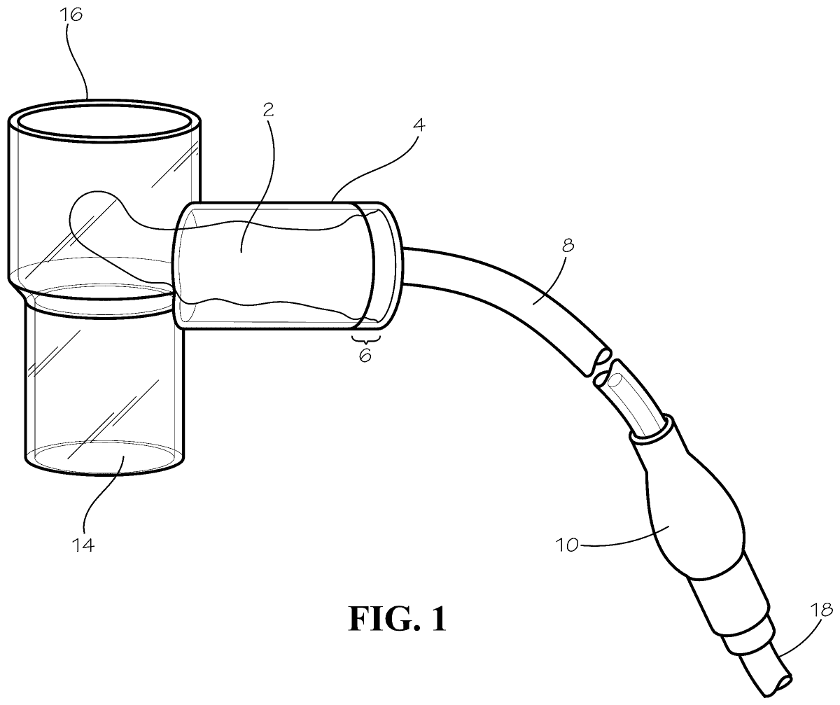

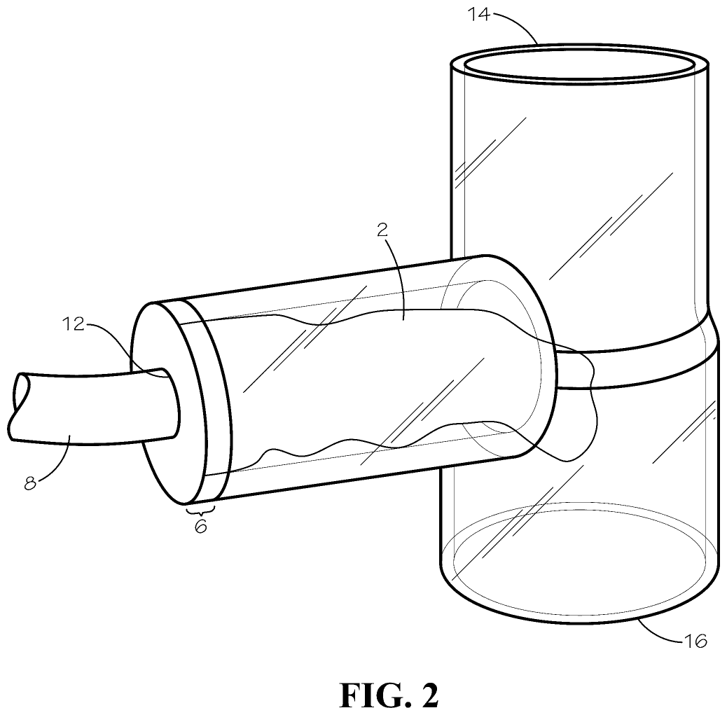

FIG. 1 is a diagram showing a side view of an embodiment of a PEEP retention valve and balloon 2 deflated. ETT port 16 for attachment and connection to an ETT and ventilator circuit port 14 for attachment and connection to a ventilator circuit. Balloon 2 is housed in balloon housing 4. Cap 6 seals and affixes the balloon intake of balloon 2 around the periphery of and within balloon housing 4 for balloon inflation from one way inflation tube 8 (one way inflation tube 8 connected to balloon inflation port 12 in FIG. 2). The airflow chamber is the air flow path from ETT port 16 and the ventilator circuit port 14. As shown, deflated balloon 2 encroaches into the air flow chamber between ETT port 16 and ventilator circuit port 14 through a chamber connection between balloon housing 4 and the air flow chamber between ETT port 16 and ventilator circuit port 14. Balloon 2 may be inflated by providing air through air intake 18 connected to one way valve 10 connected to one way inflation tube 8. When balloon 2 is deflated as shown in FIG. 1, air may flow between ETT port 16 and ventilator circuit port 14.

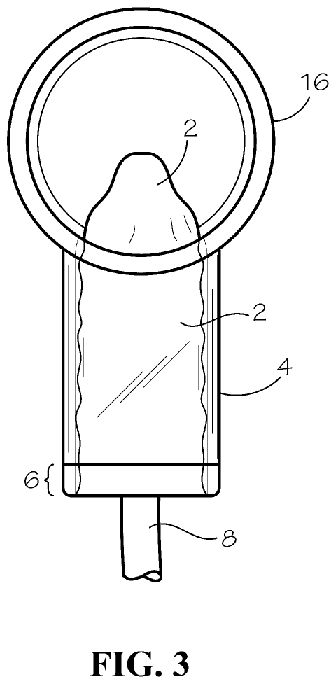

FIGS. 2 and 3 are diagrams showing a perspective view and top view looking into ETT port 16, respectively.

As shown a tube is connected to balloon inflation port 12 for balloon inflation. (e.g., using a tube connected to balloon inflation port 12 at one of its ends and a unidirectional flow valve such as a duck bill valve at its other end to inflate the balloon).

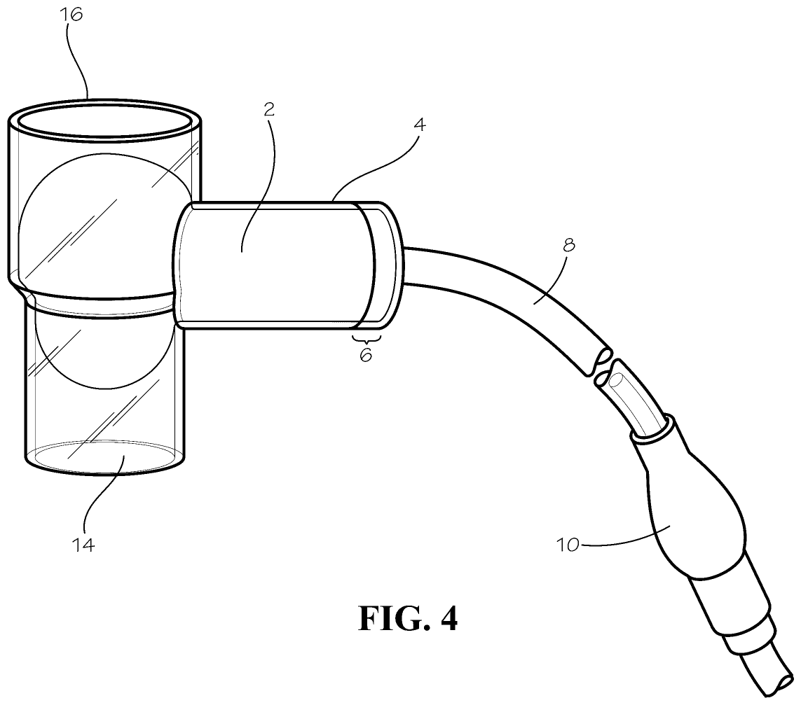

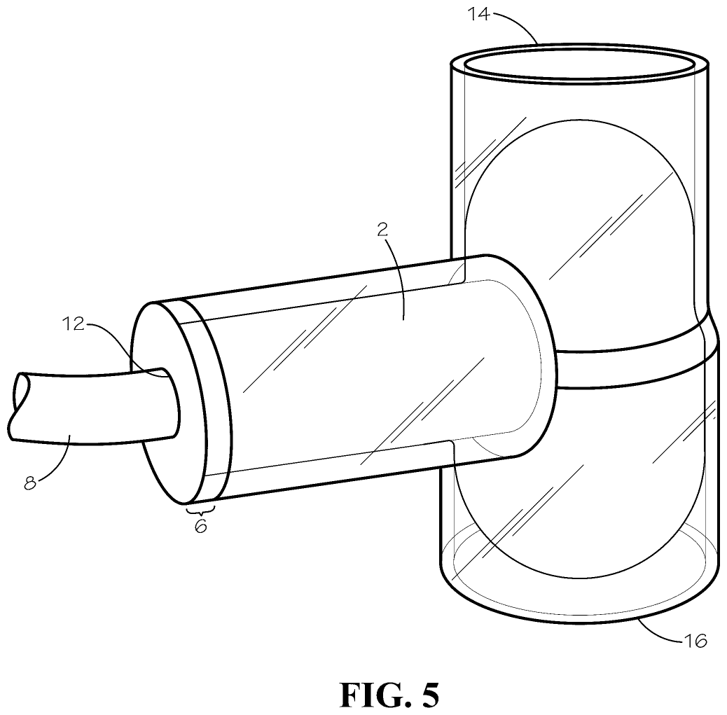

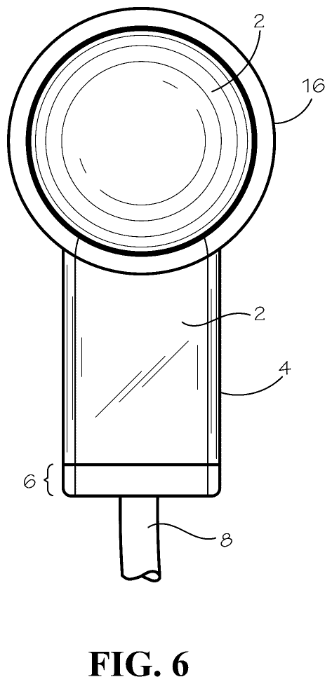

FIGS. 4, 5, and 6 are diagrams showing the PEEP retention valve of FIGS. 1, 2, and 3 with balloon 2 inflated and air flow chamber between ETT port 16 and ventilator circuit port 14 occluded such that air may not pass in or out of ETT port 16. As the balloon is inflated with air, it begins to occupy and expand into the chamber (balloon housing 4 and the air flow chamber between ETT port 16 and ventilator circuit port 14). Eventually the balloon is inflated enough such that it forms an air seal in the chamber and maintains positive end expiratory pressure in an ETT connected to the ETT port as shown in FIGS. 4, 5, and 6 (connected ETT not shown). The sealing points between the balloon and the balloon housing and the air flow chamber between the ventilator circuit port and the ETT port are formed and maintained by the force of the air pressure within the balloon pressed against the balloon housing and the air flow chamber.

The PEEP valve of FIG. 1 is a cylindrical T-piece adapter that has three ports. ETT port 16 is the port that connects to the ETT and it may have dimensions of an 18 mm outside diameter and a 15 mm inside diameter. Ventilator circuit port 14 attaches to the ventilator circuit and may have dimensions of a 15 mm outside diameter and a 12 mm internal diameter. Adapters are readily available to provide diameter adjustment for secure connection and attachment for both ports to ETT and ventilator circuits.

Balloon housing 2 is shown housing a deflated balloon in FIG. 1 and an inflated balloon in FIG. 4. Balloon housing 2 may have dimensions of a 15 mm outside diameter and 12 mm inside diameter.

The balloon may be made of neoprene or silicone (any non latex rubber). Generally, the balloon shape has a finger on a glove shape and may be rolled up over the end of the balloon housing to affix the balloon by the balloon intake in the balloon housing.

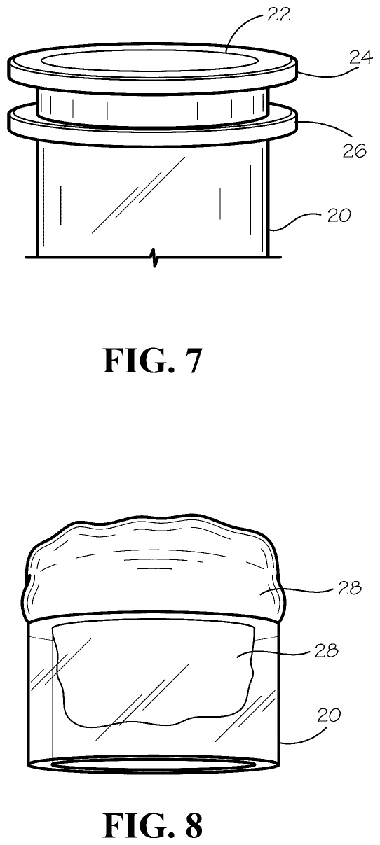

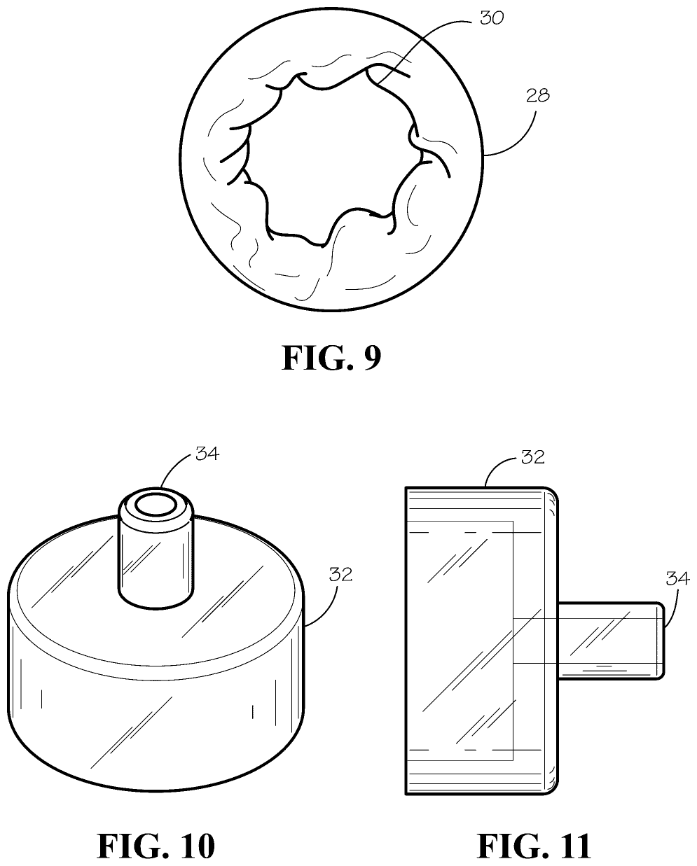

Balloon securing features such as external ribs (e.g., 2 mm ribs adding 2 mm to largest external diameter) may be provided to securely affix the balloon by the balloon intake to the balloon housing FIG. 7 is a diagram showing a side view of balloon housing 20 showing balloon port 22 and external ribs 24 and 26. FIG. 8 is a diagram showing a side view of balloon housing 20 showing the air intake of balloon 28 rolled/stretched over ribs 24 and 26 and the fillable body of balloon 28 in balloon housing 20. FIG. 9 is a diagram showing a top view looking into balloon port 22 of balloon housing 20 showing the air intake of balloon 28 rolled/stretched over ribs 24 and 26 and the fillable body of balloon 28 in balloon housing 20. Adhesives may be used to secure and provide an airtight seal of air intake of balloon 28 rolled/stretched over ribs 24 and 26. Alternatively, air intake of balloon 28 may rest between ribs 24 and 26 instead of over both as shown in FIG. 8.

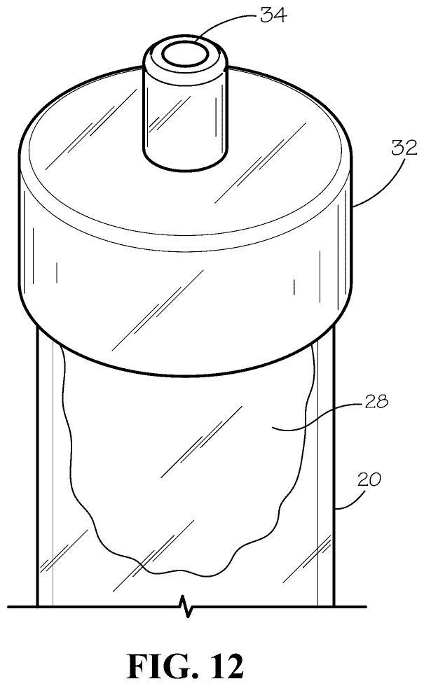

The balloon may be advantageous secured to the balloon port and affixed within the balloon housing by a balloon cap. FIG. 10 is a diagram showing a perspective view and FIG. 11 is a diagram showing a side view showing cap 32 having balloon port 34 for connecting to a one way air valve. FIG. 12 is a diagram showing a perspective view of cap 32 having balloon port 34 on and over balloon housing 20 and the air intake of balloon 28 rolled/stretched over ribs 24 and 26 of FIG. 8. After the air intake of balloon 28 is rolled/stretched over ribs 24 and 26 and the fillable body of balloon 28 is placed in balloon housing 20 (e.g., with the balloon tip flush with the outside diameter of the airflow chamber), cap 32 is then placed and affixed over the balloon and balloon port. Adhesives may be used to secure and provide an airtight seal of cap 32 to the air intake portions of balloon 28 rolled/stretched over ribs 24 and 26. Cap 32 may also have a lip on its inner chamber that will snap into place beneath the second rib to provide an airtight seal. In other words, once the balloon is placed in the balloon port and the balloon intake end is placed over the ribs a cap is placed on the balloon port.

The fillable body of balloon 28 in balloon housing 20 may be filled by forcing air into balloon port 34. The cap has a tube extending from the center of it. Tubing connected to balloon port 34, for example tubing having a 4 mm outside diameter and a length of 10 mm long with a screw end tip open to the balloon for connection to a one way valve. For use as both an air intake and air evacuation port, balloon port 34 may advantageously connected to tubing having a unidirectional flow valve (duck bill) having a screw type connector that attaches that allows 5-10 cc's of air to be introduced into the balloon, for example using a 10 cc syringe inserted/attached to the unidirectional flow valve. As a safety feature that allows air to be quickly evacuated from the balloon, to evacuate air from a filled balloon, the tubing connected to balloon port 34 may be readily removed thus releasing the air from balloon 28.

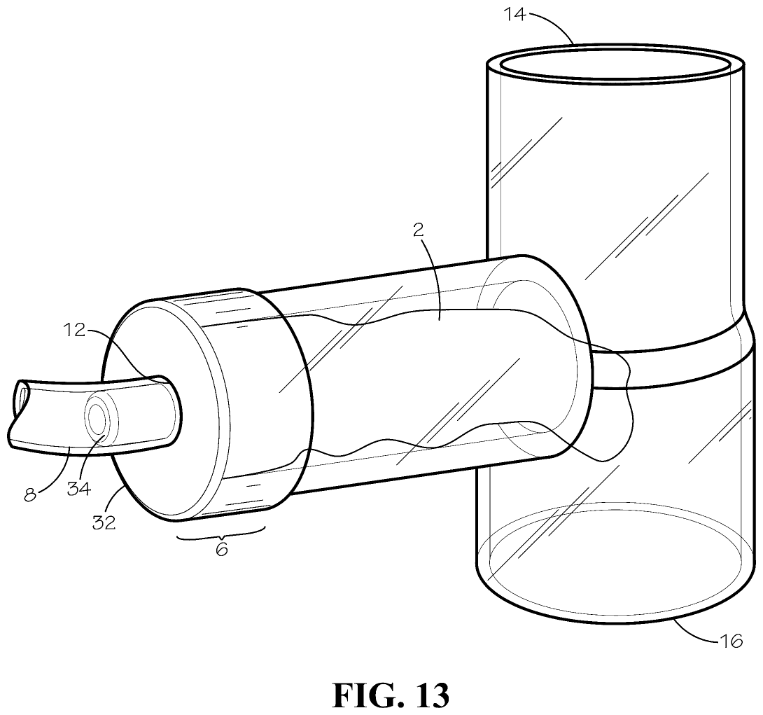

FIG. 13 is a diagram showing a perspective view of the cap 32 on the PEEP retention valve of FIG. 1 and providing the sealing of the air intake of balloon 2 and balloon port 34 for inflation of balloon 2 via one way inflation tube 8.

FIG. 14 is a diagram showing a perspective view of a PEEP retention valve consistent with that of FIG. 1 having ETT port 42, balloon port 44 and attached to ventilator circuit WYE 46 at ventilator circuit port (not shown).

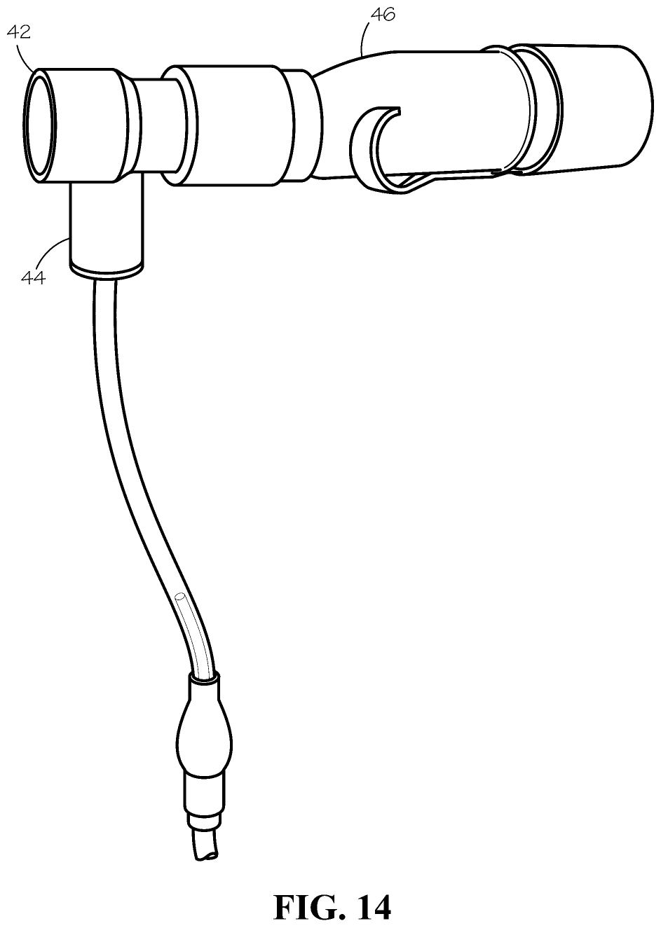

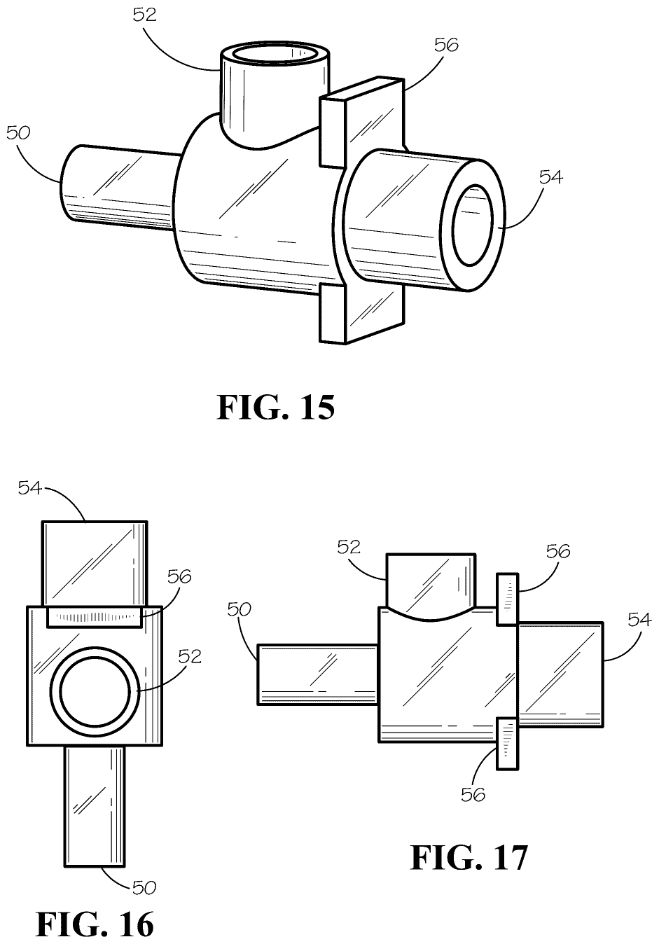

FIGS. 15, 16, and 17 are diagrams showing perspective, top, and side views of a PEEP retention valve adapter consistent with the PEEP retention valve of FIG. 1 having ETT tube 52 and balloon port 52 and with tapered tube port 54 for attachment to a ventilator circuit. Grip tabs 56 are used to disengage a ventilator circuit from tapered tube port 54. Tapered ETT tube port 54 may have teeth to prevent PEEP retention valve adapter from inadvertently being dislodged from the ETT.

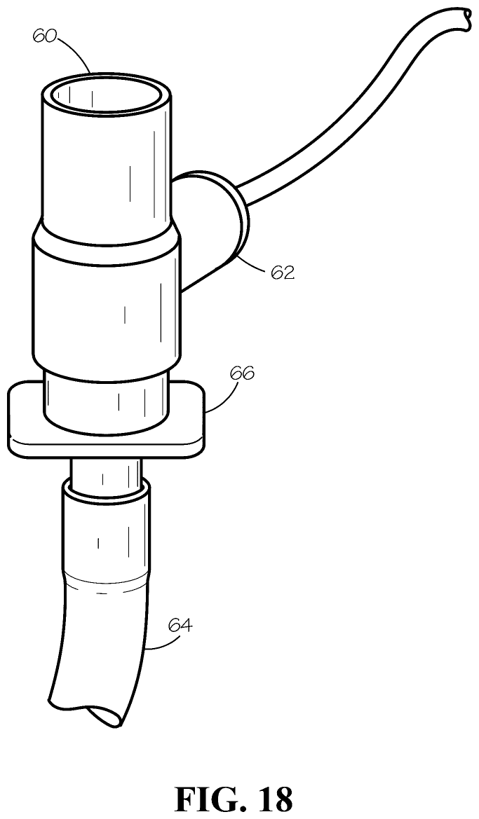

FIG. 18 is a diagram showing a side perspective view of the PEEP retention valve of FIG. 1 connected to ETT 64 having grip tabs 66. Balloon port 62 and circuit port 60

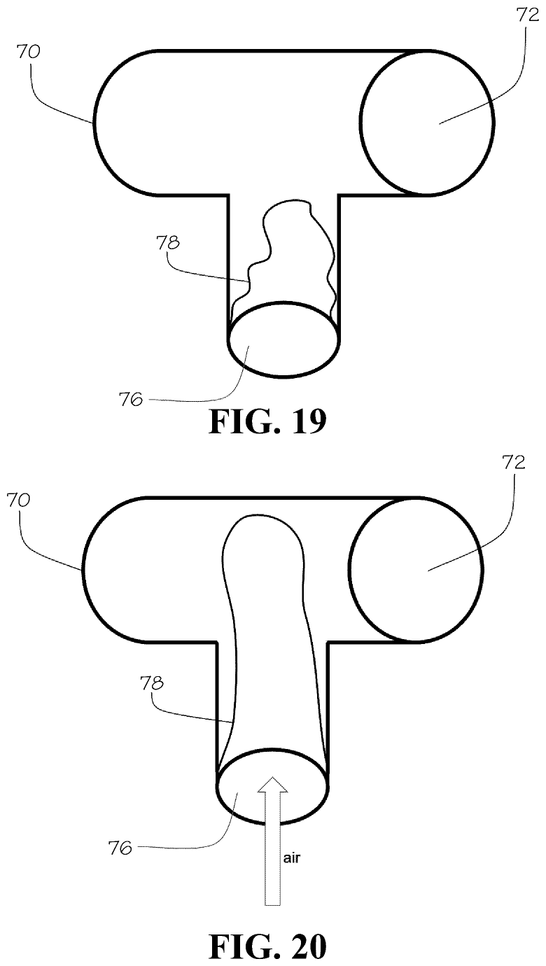

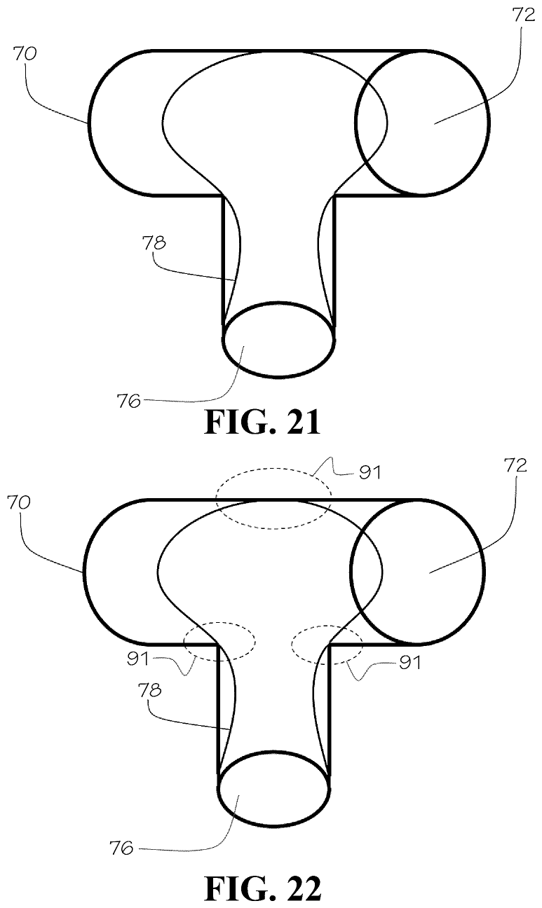

FIGS. 19 through 22 are diagrams showing the flow of inflation of balloon 78 in a positive end expiratory pressure retention valve having ventilator circuit port 70, ETT port 72, and balloon port 76. The fixation of the air intake of balloon 78 at sealing points a′ in FIG. 22 are shown as seals for descriptive purposes, in operation the balloon fills and expands into the cavity depending on the balloon material, size, air pressure, and cavity shape.

The foregoing description of the exemplary embodiments is provided to enable any person skilled in the art to make or use the claimed subject matter. Various modifications to these embodiments will be readily apparent to those skilled in the art, and the generic principles defined herein may be applied to other embodiments without the use of the innovative faculty. Thus, the claimed subject matter is not intended to be limited to the embodiments shown herein but is to be accorded the widest scope consistent with the principles and novel features disclosed herein.

What is claimed is:

1. A positive end expiratory pressure retention valve comprising:
a tube house forming an air flow chamber between a first port and a second port;
said first port for connecting to a medical air supply tube;
said second port for connecting to a medical air supply source;
a balloon house connected to said tube house and housing a balloon in a balloon chamber connected to said air flow chamber whereby said balloon occludes said air flow chamber when inflated;
the air intake of said balloon sealed to a balloon port; and,
and a one way air tube connected to said balloon port for inflating said balloon.

2. The pressure retention valve of claim 1, wherein said medical air supply tube is an endotracheal tube.

3. The pressure retention valve of claim 1, wherein said medical air supply source is a ventilator.

4. The pressure retention valve of claim 1, wherein said medical air supply source is a ventilator circuit of a ventilator.

5. The pressure retention valve of claim 1, wherein said one way air tube is connected to a one way air valve.

6. The pressure retention valve of claim 1, further comprising a balloon house cap sealing the air intake of said balloon and providing said balloon port.

7. The pressure retention valve of claim 6, wherein said one way air tube is connected to a one way air valve.

8. The pressure retention valve of claim 1, wherein said tube house, balloon house, and balloon port are made of plastic.

9. The pressure retention valve of claim 1, wherein said tube house, balloon house, and balloon port are made of silicone.

10. The pressure retention valve of claim 1, wherein said tube house, balloon house, and balloon port are made of neoprene.

11. The pressure retention valve of claim 1, wherein said balloon is made of silicone.

12. The pressure retention valve of claim 1, wherein said balloon is made of neoprene.

13. A method for maintaining positive end expiratory pressure, the method comprising:
inflating a balloon in a pressure retention valve through a one way air valve, said pressure retention valve having:
a tube house forming an air flow chamber between a first port and a second port;
said first port connected to a medical air supply tube;
said second port connected to a medical air supply source;
a balloon house connected to said tube house and housing a balloon in a balloon chamber connected to said air flow chamber whereby said balloon occludes said air flow chamber when inflated;
the air intake of said balloon sealed to a balloon port; and,
and a one way air tube connected to said balloon port for inflating said balloon;
said inflation occluding said air flow chamber; and,
removing said medical air supply source from said second port of said pressure retention valve.
